(19) United States
(12) Patent Application Publication
(10) Pub. No.: US 2008/0015457 A1
(43) Pub. Date: Jan. 17, 2008

(54) Device for Monitoring Respiratory Movements

(76) Inventor: Carlos Daniel Silva, Buenos Aires (AR)

Correspondence Address: NORRIS MCLAUGHLIN & MARCUS, P.A., P O BOX 1018, SOMERVILLE, NJ, 08876, US

(21) Appl. No.: 11/834,287

(22) Filed: Aug. 6, 2007

Related U.S. Application Data
(63) Continuation-in-part of application No. 10/534,166, filed on May 5, 2005.

(30) Foreign Application Priority Data
Nov. 7, 2002 (AR) P020104280

(51) Int. Cl. A61B 5/08

(52) U.S. Cl. 600/534

(57) ABSTRACT

The present invention relates to a monitor respiration movements device to be used on humans and also on animals for controlling the respiration movements and to control the apnea periods on infants, wherein the device reduces the mortality rate caused by the sudden instant death syndrome (SIDS), wherein the device comprises an accelerometer and a micro controller, with the accelerometer including a motion detector and a plurality of output plugs, the micro controller includes a plurality of input sockets, and wherein the plurality of output plugs are connected to the plurality of input sockets and the micro controller includes signal outputs which are connected to an alarm.

### CROSS REFERENCE TO RELATED APPLICATIONS

The present application is a continuation-in-part application of U.S. patent application Ser. No. 10/534,166, filed May 5, 2005, the disclosure of which is incorporated herein by reference and from which priority benefit is claimed.

### BACKGROUND OF THE INVENTION

1. Field of the Invention

The present invention relates to a device for monitoring respiratory movements for controlling apnea periods both in humans and in animals. Furthermore the present invention is related to a device for reducing the mortality rate caused by the sudden instant death syndrome (SIDS).

2. Description of the Prior Art

The study of respiratory movements and lung capacity is considered a major subject in the medicine filed. For such studies several methods and technologies have been used gathering every bit of information that could lead to a better detection and diagnosis of lung and respiratory dysfunction.

The use of spirometers dates since the 17th century. The spirometers measure the lung capacity volume of a human being but they cannot measure the residual function capacity of the lungs. Another device used in such medical field is the pletismograph. The pletismograph allows achieving better and more complex studies of the abovementioned respiratory and lung disorders.

However, both the spirometers and the pletismographs results obtained by the use of such devices generated a limited result based on the data obtained by those devices.

Further, the use of transducers, the refurbishing of the signals obtained and the digital analysis of data, gave place to a better respiration monitoring by implementing the new technology to the common devices. Nevertheless there is still some situations where the respiration monitoring is not fully developed leading to several holes in that field. For example, while the removing from a patient the tubes from a life support machine, the patient is exposed to a tremendous risk where his body could not be prepared to breathe by it self. Since the moment the tubes of the life support machine are removed from the patient, there is no more monitoring of the patient so the doctors can not tell whether the patient is able to breath by him self not until a few vital seconds and even minutes had passed by, wherein some times those seconds or minutes could lead so death.

On the other hand, the Sudden Infant Death Syndrome (SIDS) is a medical disorder that claims the lives of many babies from one month to one year of age each year. Once known as crib death, these infant deaths remain unexplained after all known causes have been ruled out through autopsy, death scene investigation, and medical history. SIDS affects families of all races, religions, and income levels. It occurs during sleep, and strikes without warning. Its victims appear to be healthy. Neither parents nor doctors can tell which babies will die. The first year of life is a time of rapid growth and development when any baby may be vulnerable to SIDS.

According to some recent theories, the baby stops breathing because when sleeping starts to dream as if he were still within the mother's body where no breathing is necessary. If this is so the immediate solution would be to weak up the baby to restore breathing. Then, the solution for this particular cause or even upon the breathing interruption due to any other reason, would be to weak up the baby.

The reason why SIDS happens to babies is still a mystery to find out, although researchers are making great progress in identifying deficits, behaviors, and other factors that may put an infant at higher risk. Scientists are exploring the development and function of the nervous system, the brain, the heart, and breathing and sleep patterns, body chemical balances, autopsy findings, and environmental factors. Researchers from several universities have, in fact, isolated a neurochemical defect in a portion of the brain of SIDS victims that controls the infant's protective responses to changes in oxygen and carbon dioxide levels. It appears likely that SIDS may be caused by some subtle developmental delay, an anatomical defect or functional failure. SIDS, like other medical disorders, may eventually have more than one explanation and more than one means of prevention. This may explain why the characteristics of SIDS babies seem so varied.

There are several technologies known in the art that monitors the respiration movements, some of them measure the pressure, some detects the electrical resistance variation taken from a transducer, while other technologies sense the respiration movements of the human body.

The problem for measuring the pressure values obtained by the respiration movements monitored from a human being or animal is to obtain reliable references to perform the tests. To overcome the mentioned problem there are two major technologies used to monitor the apnea in babies. The first one uses a pressure transducer, which is placed under the mattress to monitor the baby's respiration movements. The second technology consists in adhesively attaching a balloon on the baby's abdomen, connecting said balloon to a pressure transducer. The variations in the electrical resistance must be detected by the use of a belt placed around the baby's body.

When using a transducer under the mattress, as mentioned before, the changes of pressure produced by the respiration movements are partially absorbed by the mattress itself therefore the reading obtained by the transducer as not quite accurate. In the event that a balloon is attached to the baby's abdomen, the reading a rally accurate compared to the technology described above, but since the balloon has to be attached to the baby's abdomen by an adhesive material, said adhesive material prevents from using the balloon in babies for more than 8 month, since skin reactions may appear leading into a rash and making the baby very uncomfortable. Furthermore, while monitoring the baby's respiration movements one must avoid the use of wiring in such devices since no only the baby tends to play with the wiring and could lead to a malfunction of the equipment but also it could represent a big danger to him due to risk of choking.

Additionally, the monitoring in animals is still under major development since there are no new methods or technologies applied in this field. The monitoring of the respiration movements in animals such as in stallions, and the like has increased significantly. Nowadays there are several veterinarian therapies to be performed on animals, being those therapies very similar to the ones used on humans, including the use of life support devices. However the monitoring techniques are still very limited. One of the major problems involved in the monitoring techniques is the difficulty of placing the wiring, catheter, sensors and the like in the animal.

U.S. Pat. No. 6,472,988 refers to a system for monitoring wearers of respiratory equipment, such as firemen, wherein the alarm is actuated when an alteration in pressure, temperature, movement, etc. is detected. Thus, this is a system detecting big movements like the ones of a person walking, running and the like, but incapable of detecting the small, almost undetectable, breathing movement of a sleeping baby. In addition, the system includes an alarm operating in a normally “off” status that is activated upon any of the above mentioned alterations, Therefore, upon the failure of any part of the circuit, such as recording system, transmission system, etc. the alarm fails to actuate.

As stated in column 9, lines 18-22, “Antenna 62 may be, for example, a lambda/4 line antenna which, at this frequency, has a length of approximately 17 cm. To keep the power consumption of mobile part 21 as low as possible, UHF transmitter 60 is activated only when needed.” If UHF transmission is activated only when needed, it is clear that there is no continuous monitoring of “Mobile unit” 21, so the system could fail or could be so far of the “Base station” 20 in a manner that station 20 could read that no data is sent because all is in order. The system operates upon the reception of data acquisition, therefore, the alarm is not activated upon a failure of the system. In addition, the term “Power supply” 105 shows that there is no direct connection of the alarm in order to be actuated upon a general failure.

In addition to the foregoing, the U.S. Pat. No. 6,472,988 discloses a complex equipment having a base station with plurality of mobile units to be disposed on a breathing apparatus. This is not a portable, small and compact monitoring apparatus that can be worn in the garment of a baby.

U.S. Pat. No. 6,254,551 discloses an apparatus for monitoring vital functions and for processing the results, comprising a sensor arrangement to be fitted onto the user's chest by means of a belt or band 9. It is apparent that this system can not be used in a baby's body when slipping because the belt will be uncomfortable for the baby's dream as an obstacle to the normal breathing.

To overcome the drawbacks aforementioned there is a need for a respiration movement monitor that can be easily handled, compact, reliable in the reception of signals, and that will not interfere with the respiration movements of the human or animal.

### SUMMARY OF THE INVENTION

It is therefore one object of the present invention to provide a monitoring respiratory movements device to be used both in humans and animals for controlling the respiration movements and specially the apnea periods in infants to reduce the mortality rate caused by the sudden instant death syndrome (SIDS).

It is still another object of the present invention to provide a monitoring respiratory movement device for improving the monitoring techniques and methods used in the veterinary filed for monitoring stallions and the like.

It also another object of the present invention to provide a monitoring respiratory movement device that uses an accelerometer as a sensor, since there are acceleration motion in the respiration movements that can be monitored. This accelerometer(s) is(are) placed in a silica chip using nanotechnology, thus resulting in a device of really low mass and according the particular arrangement and position of the one or more accelerometers, thus providing a very sensitive device to detect the acceleration vector in the respiration movements. By use of a micro controller and with software associated, the device can perform every necessary function to fashion the signals received from the accelerometer, transmit them and eventually releasing the alarm from its inhibition because the alarm means are normally activated but inhibited or disabled by the microcontroller under normal operation conditions, namely while normal breathing is being detected.

It is a further object of the present invention to provide a monitor respiratory movement device to be used on both humans and animals for controlling the respiratory movements wherein the devices comprises an accelerometer, a micro controller, said accelerometer includes a motion detector and a plurality of output plugs, said micro controller includes a plurality of input sockets; wherein said plurality of output plugs are connected so said plurality of input sockets and the micro controller includes signal outputs which are connected to an alarm means.

It is another object of the present invention to provide a device for monitoring breathing of a wearer, the device comprising:
a box for wearing in the wearer s garment, the box being free of any means for retaining the box against the wearer's body;
an electronic circuit located into said box, wherein the circuit comprises:
at least one accelerometer arranged into said box in a manner to sense a component, or variation of the component, of a gravity acceleration vector due to angular movements of the box,
a micro controller for detecting said component, or variation, sensed by the accelerometer, and
alarm means connected to the microcontroller in a manner that the alarm means activates at least when the micro controller does not detect any component or variation in the component, of the gravity vector sensed by the accelerometer during a predetermined period of time.

The above and other objects, features and advantages of this invention will be better understood when taken in connection with the accompanying drawings and description.

### BRIEF DESCRIPTION OF THE DRAWINGS

The present invention is illustrated by way of example in the following drawings wherein:

FIG. 1 shows a block diagram of the electronic circuit of the device of the present invention;

FIG. 2 shows the electronic circuit of the device of the present invention;

FIG. 3 is an embodiment of the electronic circuit of the device of the present invention;

FIG. 4 is a perspective view taken from the back of a box or holder for the device of the present invention;

FIGS. 5, 6 are schematic views of a baby with the device of the invention placed onto his trunk with the device moving angularly along angle φ under the breathing of the baby.

### DESCRIPTION OF THE PREFERRED EMBODIMENTS

As stated before, even though the present invention can be used either for human or animal respiration monitoring, the following description is based exclusively in the monitoring of respiration movements of infants, and specially in one month to one year old babies. Therefore, the following example should not be considered as a limit to the scope and spirit of the present invention.

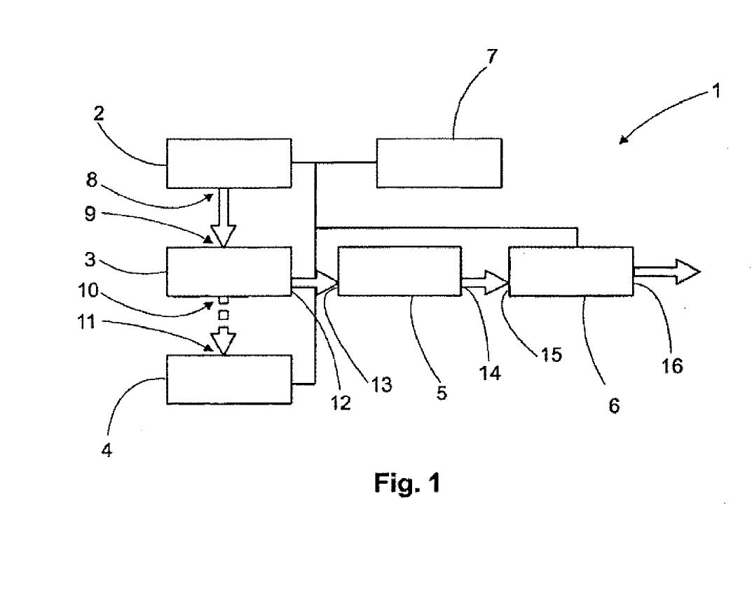

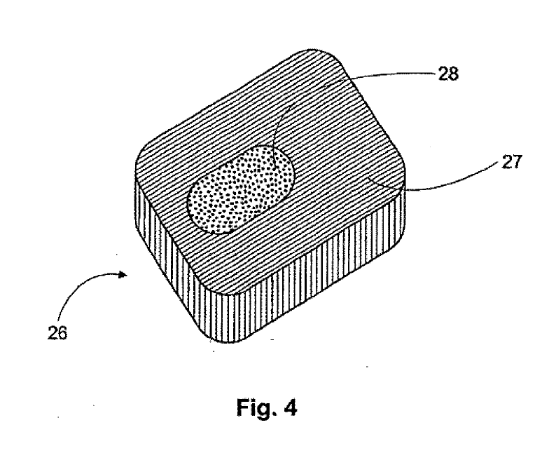

Now referring in detail to FIG. 1, the monitoring respiration movement device is defined by an electronic circuit generally described with the reference number 1. Said electronic circuit 1 comprises an accelerometer 2 including a motion detector, such as the one showed in FIG. 4. A micro controller 3, alarm means 4, instant acceleration transmission means defined by a series/parallel converting module 5 and a signal transmission module 6, and a feeding source 7 are also connected to the electronic circuit 1.

The accelerometer 2, being in this embodiment an ADXL202, is an accelerometer of a very high sensitivity and a very low mass. These characteristics allow to not interfering with the normal respiratory movements of the infant. The accelerometer 2 includes digital signal outputs 8 connected to respective inputs 9 of the micro controller 3. The micro controller 3 includes an output 10 from where the alert signals are sent to the input 11 of the alarm means 4.

The micro controller, being in this embodiment a PIC16F87 model, has implemented every necessary function to read the signals of the output 8 of the accelerometer 2. According to the software used in the micro controller 3, several signals from output 12 can be sent to the inputs 13 of the series/parallel converting module 5 and then from the outputs 14 to the inputs 15 of the transmission module 6, to send from the outputs 16 the signals from the accelerometer 2 towards others signal processing devices. Said transmission of signals can be either galvanic or wireless depending on the transmitting module to be used.

A processing device can be employed as a computer where the processing of the signals will be subject to the software used in the same. If the processing it taken in a laboratory with animals, a galvanic transmission can be safely used. The data should be input trough one of the serial ports (RS232) in the computer. For a wireless transmission of the signals an electromagnetic signal can be easily used, being the most common used signals the radio frequency signals and the infrared signals. However the device of the present invention can be equipped with ultrasound equipment, being these methods of transmitting the signals obtained from the accelerometer 2 not to be considered as limiting the scope of the present invention.

The converting module 5 included in the micro controller 3, sends the signals to the transmission module 6. In this embodiment the transmission module 6 comprises two integrated circuits defining an encoder such as a MCP2150 which encodes the received signal in such a fashion that can be transmitted by an infrared transmitter 6′ (e.g. TFDS4500). The infrared transmitter 6′ is an IrDA certified transmitter which transmits data at the speed of 115.2 Kb/sec.

By means of the converting module 5 the parallel n bits signals delivered by the micro controller 3 are converted to a series of n bits, which are added to perform the necessary control tasks. In this embodiment the accelerometer 2 has a 12 bits resolution, however only the more significant 8 bits were used in the assays.

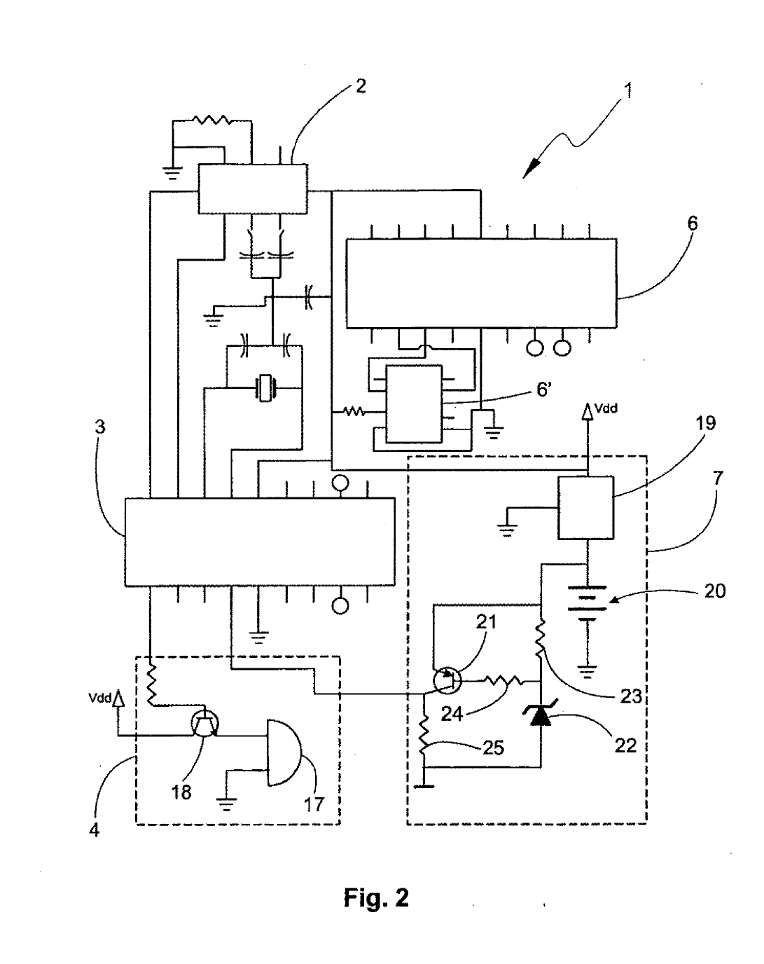

Referring now to FIG. 2 the alarm means 6 comprises a buzzer 17 connected to output 10 of the micro controller 3 through a transistor 18. The alarm means 6 can present several settings. For example, based on a multivibrating circuit and a speaker attached to it or the speaker can be replaced by a LED or even a combination of both. Still referring to FIG. 2, the power supply 7 is defined by a voltage regulator 19 such as a 78L05 voltage regulator. The voltage regulator 19 is connected to a battery 20 associated to voltage regulator circuit configured based on a transistor 21 and a Zener diode 22 with their corresponding polarization resistors 23 to 25. The circuit associated to the Zener diode and transistor 21 is for disabling the microcontroller when the tension of the battery is low, but still above the regulated tension, so that when the microcontroller is disabled a continuous alarm sound is emitted indicating that the battery charge is low.

In the event that the device of the present invention is used in humans, the power supply 7 delivers DC power required for the proper performance of the circuit from the battery 20. By doing so, the device does not need to be connected to the electric network, protecting the integrity of the human being. In addition, if any part of the system or circuit fails, the alarm is activated because it is always and directly (as shown) connected to Vdd, that is the alarm is directly fed by the battery without passing through the microcontroller.

The accelerometer 2 sends modulated signals to the micro controller 3 by means of the DMC corresponding to the instant acceleration measured in two orthogonal axes. The micro controller 3 includes software that demodulates the received signals sent in series to the transmitter module 6. The transmitter module 6 comprises the IrDA decoder and an IR transducer. In the event that the device of the present invention should be used as only an apnea monitor, the software detects the variations in the acceleration detected by the accelerometer 2. Since the microcontroller is permanently inhibiting or disabling the alarm activation, If no variations are detected in a period of T=20 seconds, the micro controller's software will interrupt such inhibition or disabling function whereby alarm 4 is able and free to emit alarm signals. Indeed, microcontroller 3 controls the base of transistor 18 to permit or inhibit the pass of current through the transistor to feed the alarm. The polarization of the transistor base is such that, by default, alarm 17 is fed from Vdd even during a failure of the microcontroller.

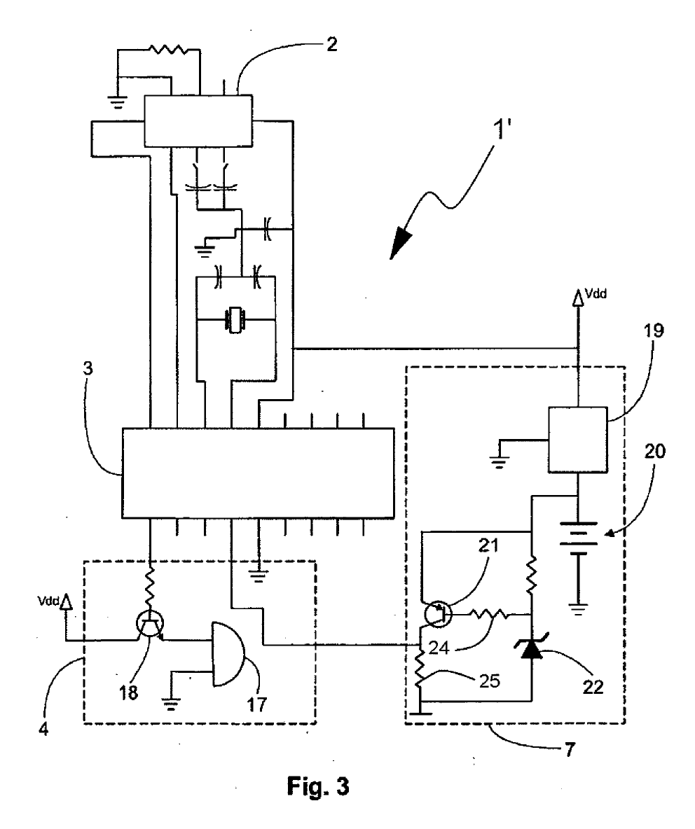

Depending on the use of the device of the present invention (e.g. as an apnea monitor for preventing SIDS), only the alarm means 4 should be connected to the micro controller 3, avoiding the use of the converting module 5 and the transmission module 6. This embodiment of the device is illustrated in FIG. 3, wherein the electronic circuit of the device is identified by the reference number 1′. Accordingly, the performance of the device using the electronic circuit 1′ is exactly the same as the performance of the device using the electronic circuit 1, except for the absence of the converting module 5 and the transmission module 6.

Referring now to FIG. 4, the geometrical configuration of the device of the present application is shown, specially the shape of the holder or box 26 inside of which either electronic circuits 1 or 1′ are housed. Further, it can be seen the motion sensors 28 placed in a wall 27 of holder 26. Box 26 defines at least one main surface to be attached to or in contact with the garments or body of the wearer and this main surface may be defined by wall 27 or the surface of the box that is opposite to wall 27. For proper function, the device must be kept in a stable and steady position with respect to the body of the wearer, either the human or animal. Preferably, the device should be placed in the trunk zone of the body where the respiration movements are more easily detected as it will be explained below. Since the device of the present invention does not need to be in touch with the skin of the human, the device can be wore over the subject's garment.

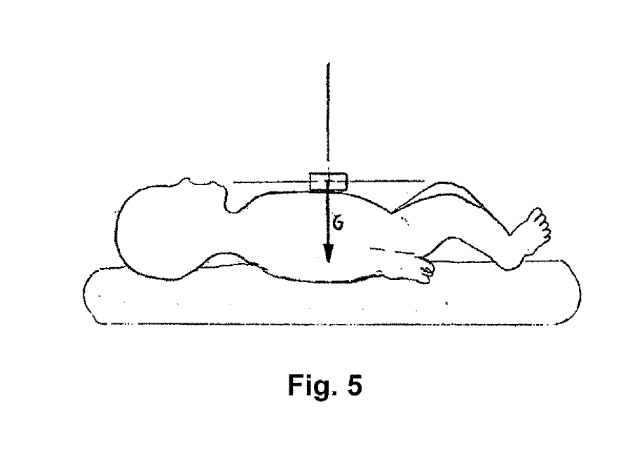

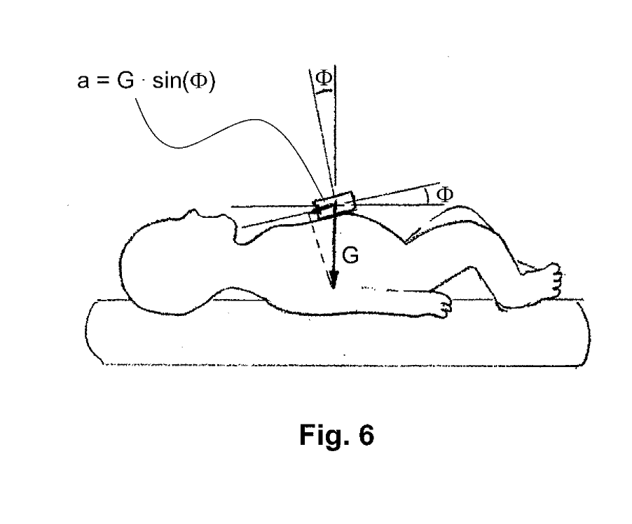

The device of the invention should be preferably placed in the body wearer, as shown in FIGS. 5, 6, according to the following teachings. Any accelerometer, while very sensitive, it is incapable of detecting very slow movements such as the one involved in breathing An accelerometer has a main sensing direction or, simply, a sensing direction to sense any acceleration in said direction. However, even if the acceleration vector to be detected extends along such direction such acceleration will not be detected if the value of same is very small.

When detecting breathing movements the following considerations must be taken into account:
the abdominal wall is moved up and down along only about 3 cm.
the breathing frequency is about 10 per minute.
inspiration/expiration rate is about ⅓.
the acceleration formula is:

$$a = \frac{2 \cdot x}{{t}^{2}}$$
wherein
X=distance of the breathing movement
t=time

By replacing the figures:

$$a = \frac{2 \cdot 3 \cdot {10}^{- 2} m}{36 {s}^{2}}$$

$$then \text{:}$$

$$a = \frac{0.06 m}{36 {s}^{2}} = 0.0016 \frac{m}{{s}^{2}} = 0.17 mg$$

An accelerometer with a very high sensitivity for detecting 0.17 mg would be so sensitive that any vibration or undesirable movement.

The solution, according to the invention, is to employ a very sensitive accelerometer, not undesirably so sensitive, but with the capacity of sensing the breathing movements by sensing a component of the gravity acceleration, that is the vertical acceleration vector, when the component appears to vary upon the inclination of box 26. This component is larger than the acceleration vector resulted from the vertical breathing movement. Since the accelerometer has a sensing direction, the way to detect such component or variation is by placing the box in a manner that when the baby breaths the box is inclined in addition to the normal up and down movement. According to the invention, the accelerometer is arranged within the box in a manner that the sensing direction is not vertical, that is, not aligned with the direction of the gravity acceleration. The sensing direction may be in any position except aligned with the vertical. In other words the sensing direction must be transversal to the main surface of the box and to any plane perpendicular to the main plane of the box.

Preferably, the sensing direction of the accelerometer will be placed perpendicular to the vertical of “g”, namely the gravity direction and more preferably, parallel to the main surface of the box. Thus, if the box is onto a table, the gravity acceleration will be measured as being cero. If the box is inclined, the appearance of a component of the gravity acceleration will be sensed or detected and this is the indication that the baby or wearer is breathing. The component of the gravity acceleration when the box is inclined, for example an angle φ, will be the value of “g” multiplied by sin φ or cos φ.

If the box is inclined, as a result of breathing, for example along an angle of 2° for each breathing movement, the acceleration will be:
a: 9.8 g×sin 2°=0.342 g,
that is a value easily detected or sensed by the accelerometer. This operation is carried out by the software housed into the microcontroller and the 2° inclination is easily obtained in any respiration movement by placing the device, preferably, in the zone or boundary between the ribs and the abdomen.

According to a preferred embodiment of the invention, the alarm means is connected to the microcontroller in a manner that the alarm means activates when the micro controller does not detect any variation sensed by the accelerometer during a predetermined period of time. More particularly, the alarm means is connected to the micro controller and to a battery, as illustrated, in a manner that if variations are being detected by the micro controller, the micro controller inhibits the activation of the alarm, if no variations are detected during a period of time the microcontroller stops such inhibition so that the alarm activates and if there is any failure in the overall device the alarm is activated directly by the battery.

Also according to the invention, the at least one accelerometer is capable of sensing variations in a component of the gravity acceleration vector, and the micro controller detects the variations in the component of the gravity acceleration vector sensed by the accelerometer. According to the invention, the box includes a main surface, such as wall 26, to be attached to the body of the wearer and the at least one accelerometer defines a main sensing plane for sensing any variation in a force vector acting transversely on the main sensing plane and wherein the accelerometer is arranged in the box in a manner that the main sensing plane of the accelerometer extends in any position other than a position parallel to the main surface of the box. More particularly, the main sensing plane of the accelerometer extends perpendicular to the main surface of the box. When the at least one accelerometer comprises two accelerometers, the main sensing plane of each accelerometer extends in any position other than parallel to each other and than a position parallel to the main surface of the box. More particularly, the main sensing planes of the accelerometers extend perpendicular to each other and perpendicular to the main surface of the box.

The positioning of the accelerometer according to the above teachings of the invention makes the accelerometer detect continuous acceleration, namely gravity acceleration, and, since the box is attached to the garment of the baby, when the baby breaths, the angular position of the accelerometer relative to the gravity acceleration vector, that is the vertical, is altered. This angular alteration causes an alteration in the detected gravity vector each time the box is inclined under the breathing movements. This difference in the detected values is the input signal in the circuit of the invention.

The good operation of the inventive device may be easily tested also according to the teachings of the invention. The circuit, preferably microcontroller 3 may include a circuit control in a manner that in the startup the alarm provides a signal, either visual or acoustic one, indicating that the overall device is correctly operating. This signal, a three “beeps” for example, is preferably distinguished from the normal acoustic and/or visual signal provided by the alarm when no breathing is detected.

In addition, the correct operation of the device may be also tested by placing the device onto a surface, such as a table, and await for 20 seconds, after which period of time the alarm must activate if the device is in order.

The invention in its broader aspects is not limited to the specific details shown and described above. Departures may be made from such details within the scope of the accompanying claims without departing from the principles of the invention and without sacrificing its advantages.

What is claimed is:

1. A device for monitoring breathing of a wearer, the device comprising:
a box for wearing in the wearer's garment, the box being free of any means for retaining the box against the wearer's body;
an electronic circuit located into said box, wherein the circuit comprises:
at least one accelerometer arranged into said box in a manner to sense a component, or variation of the component, of a gravity acceleration vector due to angular movements of the box,
a micro controller for detecting said component, or variation, sensed by the accelerometer, and
alarm means connected to the microcontroller in a manner that the alarm means activates at least when the micro controller does not detect any component or variation in the component, of the gravity vector sensed by the accelerometer during a predetermined period of time.

2. The device of claim 1, wherein the alarm means is connected to the micro controller and to a battery in a manner that if said component or any variation thereof is detected by the micro controller, the micro controller inhibit the activation of the alarm and if there is any failure in the overall device the alarm is activated directly by the battery.

3. The device of claim 1, wherein the box includes a main surface to be attached to the body of the wearer and the at least one accelerometer defines a main sensing direction for sensing an acceleration vector acting in said main sensing direction and wherein the accelerometer is arranged in the box in a manner that the main sensing direction of the accelerometer extends in any position transverse to a plane that is perpendicular to the main surface of the box.

4. The device of claim 3, wherein the at least one accelerometer comprises two accelerometers and the main sensing direction of one accelerometer extends transversely to the main sensing direction of the other acelerometer.

5. The device of claim 1, wherein said at least one accelerometer includes a motion detector and a plurality of output plugs and said micro controller includes a plurality of input sockets, wherein said plurality of output plugs are connected so said plurality of input sockets and the micro controller includes signal outputs which are connected to the alarm means.

6. The device of claim 5, wherein said micro controller comprises outputs connected to transmission means for transmitting the signals sent by the at least one accelerometer towards external processing devices for processing said signals.

7. The device of claim 6, wherein said external processing devices are computer means.

8. The device of claim 6, wherein said transmission means are instant acceleration transmission modules.

9. The device of claim 8, wherein said instant acceleration transmission modules comprise: a) a series/parallel signal converter module; b) a transmission module; wherein said series/parallel signal converter module is an integral part of the micro controller and the output sockets of the same are connected to said transmission module; the transmission module being defined by a signal codification integrated circuit and a signal transmission integrated circuit.

10. The device of claim 9, wherein said signal transmission integrated circuit is an infrared transmission device.

11. The device of claim 9, wherein said signal codification integrated circuit is an IrDA signal encoder certified.

12. The device of claim 1, wherein said alarm means is a buzzer connected to the micro controller through a transistor.

13. The device of claim 1, wherein current supply for the device is provided by an integrated regulating tension circuit connected to a DC battery associated to a circuit comprising a transistor and a Zener diode.

14. The device of claim 1, wherein said component of the gravity acceleration is the value of the gravity acceleration multiplied by the sinus of an angle along which the box is inclined during breathing of the wearer.
